(19) United States
(12) Patent Application Publication
(10) Pub. No.: US 2010/0125283 A1
(43) Pub. Date: May 20, 2010

(54) UNIVERSAL NEEDLE GUIDE

(76) Inventors: Charles Butcher, Carmel, IN (US); Jacob Flagle, New Palistine, IN (US)

Correspondence Address: RADER, FISHMAN & GRAUER PLLC, 39533 WOODWARD AVENUE, SUITE 140, BLOOMFIELD HILLS, MI, 48304-0610, US

(21) Appl. No.: 12/273,295

(22) Filed: Nov. 18, 2008

(51) Int. Cl. A61B 19/00

(52) U.S. Cl. 606/130

(57) ABSTRACT

A universal needle guide is disclosed comprising a body, a first needle aperture and a second needle aperture. The body includes an upper support portion and at least one attachment portion. The first needle aperture defines a first diameter disposed in the upper support portion. The second needle aperture defines a second diameter that is selectively alignable with the first needle aperture. The second diameter is smaller than the first diameter.

### TECHNICAL FIELD

The present disclosure relates generally to a needle guide suitable for use in a stereotactic biopsy system and more particularly to a single needle guide compatible with multiple needle gauges.

### BACKGROUND

Stereotactic breast biopsy systems are a precise, minimally invasive diagnostic tool for use in performing fine-needle aspiration and needle core biopsies of the human breast to determine whether a breast lesion is benign or malignant. Needle biopsies, unlike more invasive surgical procedures, are typically performed on an out-patient basis with local anesthesia. Stereotactic breast biopsy systems, for example, generally include an ergonomically contoured table that comfortably supports the patient in a stable, fixed position that exposes the patient's breast. Stereotactic breast biopsy systems also generally include a diagnostic imaging system for targeting the lesion to be biopsied. The location of the target lesion is used by a guidance system to guide insertion of the biopsy needle into the patient's breast tissue.

The biopsy needle used in a stereotactic breast biopsy is typically a component of a biopsy gun, which is supported by the guidance system in an appropriate holder. The biopsy needle is relatively long requiring the holder to include a support bracket to reduce deflection of the biopsy needle during deployment and use. The support bracket typically includes a needle guide at a distal end thereof through which the biopsy needle passes. The needle guide minimizes movement or deflection of the biopsy needle during a biopsy to ensure that the biopsy needle is inserted into the correct location of the patient's breast adjacent the target lesion.

Typical biopsy needle guides include an upper support portion having a hole through which the biopsy needle extends and a lower attachment portion that is removably secured to the support bracket. Each needle guide is configured with a predetermined sized hole that receives a corresponding sized needle. In other words, individual needle guides are only compatible with one needle diameter or gauge. That is, a 9 gauge needle guide is needed for 9 gauge needles and a 12 gauge needle guide is needed for 12 gauge needles. Not only does this require users to carry multiple needle guides, but it requires the user to remove the needle guide and replace it every time a different needle gauge is needed during a biopsy. Therefore, the limitations specifically minimized and/or eliminated by the present disclosure include the need for multiple needle guides due to the single gauge compatibility of current needle guides.

In addition, there exists a need to provide a needle guide that may be secured in the Z direction, as current needle guides are attached in the X and Y directions, and are therefore subject to accidental movement or repositioning of the needle guide.

### SUMMARY

A universal needle guide is disclosed comprising a body, a first needle aperture and a second needle aperture. The body includes an upper support portion and at least one attachment portion. The first needle aperture defines a first diameter disposed in the upper support portion. The second needle aperture defines a second diameter that is selectively alignable with the first needle aperture. The second diameter is smaller than the first diameter. Multiple arrangements are disclosed.

### BRIEF DESCRIPTION OF THE DRAWINGS

The features and inventive aspects of the present disclosure will become more apparent upon reading the following detailed description, claims, and drawings, of which the following is a brief description:

FIG. 1 is a perspective view of a universal needle guide.

FIG. 2 is a perspective view of another arrangement of a universal needle guide after a faceplate has been engaged.

FIG. 3 is a perspective view of another arrangement of a universal needle guide with a slidably engaged faceplate.

FIG. 4 is a perspective view of another arrangement of a universal needle guide with a slidably engaged plate.

FIG. 5 is a perspective view of another arrangement of a universal needle guide with a rotary collar.

FIG. 6A is an exploded view of a universal needle guide with a push plug actuator.

FIG. 6B is an elevational side view of a universal needle guide with a fully actuated push plug.

### DETAILED DESCRIPTION

Referring now to the drawings, illustrative embodiments of the present disclosure are shown in detail. Although the drawings represent embodiments of the present disclosure, the drawings are not necessarily to scale and certain features may be exaggerated to better illustrate and explain the disclosure. Further, the embodiments set forth herein are not intended to be exhaustive or otherwise limit or restrict the disclosure to the precise forms and configurations shown in the drawings and disclosed in the following detailed description.

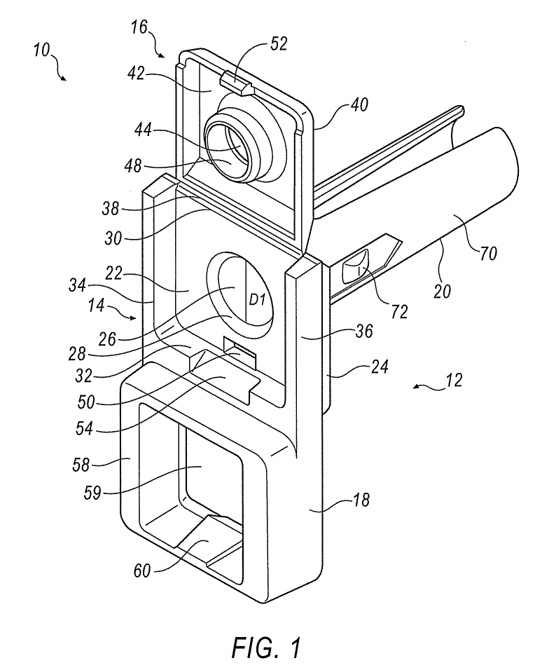

FIG. 1 illustrates a universal needle guide 10 compatible with biopsy devices having at least two needle diameters or gauges. In one exemplary arrangement, the universal needle guide 10 is a unitary member that may be formed through injection molding, although some or all components of the guide could be formed separately and assembled prior to use. While an exemplary universal needle guide 10 is shown in FIG. 1, the features illustrated in the Figure are not intended to be limiting.

The universal needle guide 10 in FIG. 1, includes a body 12 having an upper support portion 14, a faceplate 16, and at least one attachment portion 18, 20. The upper support portion 14 has a front surface 22, and a back surface 24. In one embodiment, the front surface 22 is recessed to accommodate the faceplate 16, discussed in further detail below. The upper support portion 14 also includes a first needle aperture 26 extending therethrough, defining a first diameter D1. The first needle aperture 26 may be countersunk to define a countersunk surface 28 that facilitates alignment of a biopsy needle with the first needle aperture 26 as it passes through.

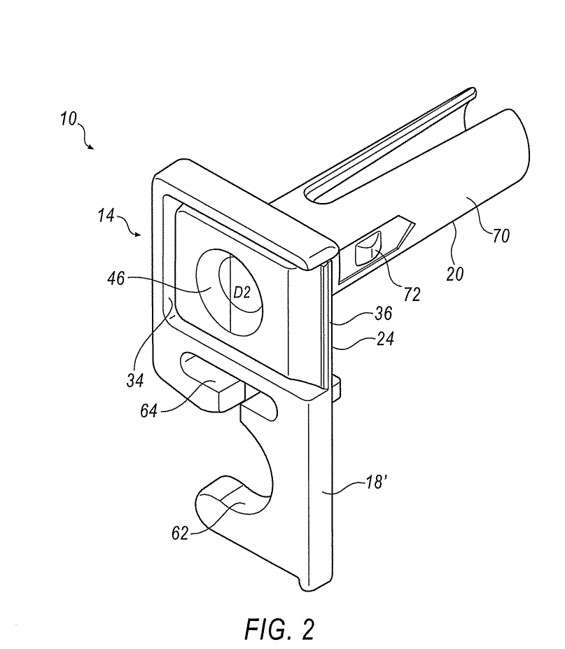

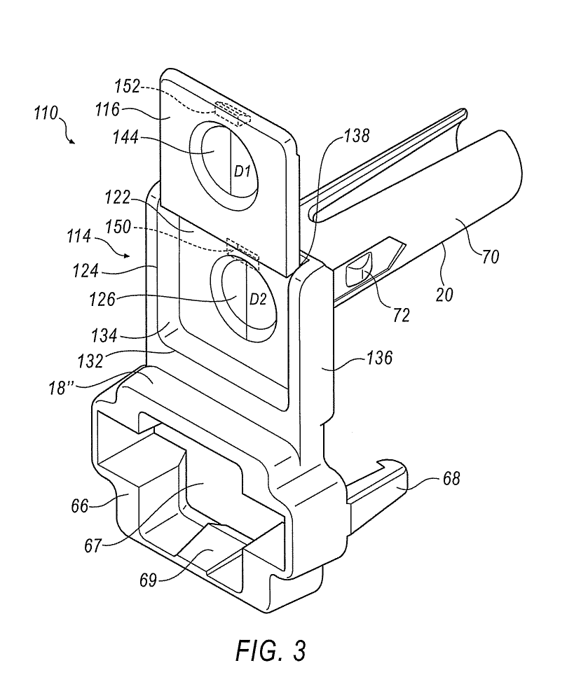

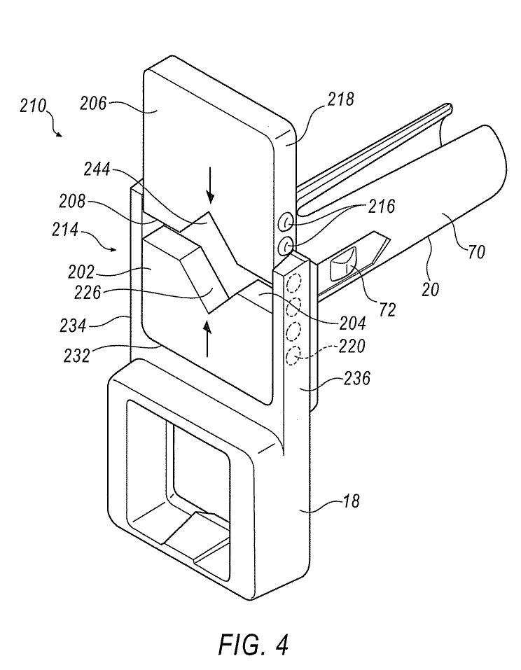

In one arrangement, faceplate 16 is pivotally attached to a surface of the upper support portion 14. For example, in FIG. 1 faceplate 16 is pivotally attached to a top surface 30 of the upper support portion 14. However, the faceplate 16 could also be attached to the bottom surface 32 or either side surface 34 and 36 of the upper support portion 14 as shown in FIG. 2. In one embodiment, the faceplate 16 is pivotally attached using a living hinge 38; however, other types of mounts and fasteners could be used. Additionally, other methods of attachment such as slidable engagement, see FIGS. 3 and 4 discussed below, are also contemplated. In FIG. 1 when the faceplate 16 is pivoted about the living hinge 38 it is configured to align with the upper support portion 14.

The faceplate 16 has a front surface 40, a back surface 42 and a second needle aperture 44 that extends therethrough, defining a second diameter D2, best seen in FIG. 2. The second diameter D2 of the second needle aperture 44 is less than the first diameter D1 of the first needle aperture 26. The second needle aperture 44 is positioned complimentary to the first needle aperture 26 so that the two apertures align when the faceplate 16 and the upper support portion 14 are mated. The second needle aperture 44 also may be countersunk to define a countersunk surface 46, best seen in FIG. 2, that facilitates alignment of a biopsy needle with the second needle aperture 44 as it passes through. The disparate diameters of the faceplate 16 and the upper support portion 14 allow the universal needle guide 10 to be selectively compatible with a larger gauge needle when the faceplate 16 is in an open position, as illustrated in FIG. 1, and a smaller needle gauge when the faceplate 16 and the upper support portion 14 are mated, as illustrated in FIG. 2.

To ensure precise alignment of the biopsy needle when the faceplate 16 and upper support portion 14 are engaged, the faceplate 16 may further include a sleeve 48 that extends from second needle aperture 44. The sleeve 48, as best seen in FIG. 1, is aligned with the second needle aperture 44 and configured to extend through the recessed front surface 22 of the upper support portion 14 and align with the first needle aperture 26 when the faceplate 16 and the upper support portion 14 are mated. Thus, sleeve 48 helps prevent improper movement of the biopsy needle by further guiding the needle through the upper support portion 14 when the second needle aperture 44 is being used. Preventing improper movement is important in any biopsy procedure because improper movement of the biopsy needle relative to the guide during positioning could result in improper positioning.

To further ensure proper alignment of the biopsy needle and to prevent improper movement of the biopsy needle, the faceplate 16 and the upper support portion 14 can be lockingly engaged. In one arrangement, as shown in FIG. 1, the universal needle guide 10 may include a detent 50 disposed in the front surface 22 of the upper support portion 14 and configured to lockingly engage a complimentary locking tab 52 disposed on the faceplate 16. The locking tab 52 and detent 50 secure the faceplate 16 and the upper support portion 14 in a mated position during use, best illustrated in FIG. 2. The upper support position 14 may also include a release notch 54 that enables a user to switch between different needle gauges while continuing to use the same needle guide 10. Although one embodiment uses a tab and detent locking mechanism other locking mechanisms could be recognized by one of ordinary skill in the art.

The attachment portion of the universal needle guide 10 may include a lower attachment portion 18 and/or a slitted cannula 20. In FIGS. 1-5 the universal needle guide includes both the lower attachment portion 18 and the slitted cannula 20 so that the guide is compatible with various stereotactic table designs. Further, use of both a lower attachment portion 18 and a slitted cannula 20 serves to lock the universal needle guide 10 in at least two different directions. However, other embodiments of the universal needle guide 10 could be manufactured for a specific table design and include only one attachment portion.

In one exemplary arrangement, the lower attachment portion 18 extends downwardly from what would be generally considered the bottom surface 32 of the upper support portion 14. In FIG. 1 the lower attachment portion 18 includes a generally rectangular periphery 58 that defines an opening 59 to receive a portion of a stereotactic table. A detent 60 may also be included within the rectangular periphery 58 to lockingly engage the lower attachment portion 18 and the stereotactic table. The lower attachment portion 18 attaching in the z direction serves to lock the universal needle guide in both the x and y axes. However, as shown in FIG. 2 and FIG. 3 other configurations may be employed. For example, in FIG. 2 the lower attachment portion 18′ includes a generally c-shaped cradling aperture 62 and a positioning slot 64. In FIG. 3, the lower attachment portion 18″ includes a periphery 66 that defines an opening 67 to accommodate various sized rails. A clip 68 may extend from the periphery 66 to lockingly engage the lower attachment portion 18″ and the stereotactic table. A detent 69 may also be included within the periphery 66 to lockingly engage the lower attachment portion 18″ and the stereotactic table.

In general, the lower attachment portion 18 is removably secured to a support bracket (not shown) that is attached to the guidance system (also not shown) used in most stereotactic biopsy systems. Due to the density of breast tissue, for example, the forces imposed on the biopsy needle are not necessarily along the needle axis, but include a component of force perpendicular to the needle axis. Thus, the lower attachment portion 18 minimizes the movement of the universal needle guide 10 relative to the support bracket (i.e., the x and y axes) when the biopsy needle is deployed.

The slitted cannula 20 is aligned with the first needle aperture 26 and extends outwardly from the back surface 24 of the upper support portion 14. The slitted cannula 20 is spring loaded or otherwise configured to be compatible with various sized support structures (not shown) disposed on stereotactic biopsy tables and serves to retain the universal needle guide 10 in the z direction. The outer surface 70 of the slitted cannula 20, best seen in FIG. 2, has a release mechanism 72 disposed thereon. To remove the slitted cannula 20, once engaged with a stereotactic biopsy table, the release mechanism 72 can be moved in a generally forward direction. Moving the release mechanism 72 causes the slitted cannula 20 to narrow and release from the stereotactic biopsy table. The needle guide 10 can then be removed and disposed.

The location of the slitted cannula 20 also aids in preventing deflection of the needle during positioning because after the biopsy needle is aligned with the first needle aperture 26 or second needle aperture 44, depending on the needle gauge being used, the biopsy needle further extends into the slitted cannula 20.

FIG. 3 illustrates another embodiment of a needle guide 110. Needle guide 110 includes a faceplate 116 that is slidably engaged with an upper support portion 114. In one embodiment, the front surface 122 of the upper support portion 114 is recessed defining a periphery 124 of the upper support portion 114. In FIG. 3 the periphery 124 defines the bottom surface 132 and two side surfaces 134 and 136 of the upper support portion 114 leaving the top surface 138 open to accommodate the sliding faceplate 116. Other embodiments could include a periphery 124 defined by the top surface 138, bottom surface 132, and one of the side surface 134 or 136 such that the faceplate 116 is slidably engaged with the remaining side surface. In the arrangement shown in FIG. 3, the faceplate 116 is free to slide along the peripheral sides 134 and 136 of the upper support portion 114 such that the first needle aperture 126 and the second needle aperture 144 align when the faceplate 116 and the upper support portion 114 are slidably aligned. The two side surfaces 134 and 136 may include rails to accommodate the sliding faceplate 116.

In FIG. 3 the universal needle guide 110 may include a flexible detent 150 (shown in phantom) disposed in the bottom surface 132 or a top surface 138 of the upper support portion 114 and is configured to lockingly engage a complimentary locking tab 152 disposed on the faceplate 116. The locking tab 152 and detent 150 secure the faceplate 116 and the upper support portion 114 in a mated position during use to prevent improper movement of the biopsy needle during positioning within a patient. Although one embodiment uses a tab and detent locking mechanism, other locking mechanisms could be recognized by one of ordinary skill in the art.

FIG. 4 is another embodiment of a universal needle guide 210 having at least one sliding plate. In FIG. 4 an upper support portion 214 is defined by a bottom surface 232 and two side surfaces 234 and 236. The first plate 202 is positioned within the upper support portion 214 such that the first plate 202 abuts the bottom surface 232 and the two side surfaces 234 and 236, but does not extend the height of the side surfaces 234 and 236. The first plate 202 also has a substantially v-shaped notch 226 centered on a top surface 204 of the first plate 202 and extending downwardly therefrom. A second plate 206 may also have a complimentary v-shaped notch 244 centered on the bottom surface 208 of the second plate 206 and extending upwardly therefrom. However, it is understood that the v-shaped notch 244 of the second plate 206 may be omitted. The second plate 206 is slidably engaged with the side surfaces 234 and 236 of the upper support portion 214.

The second plate 206 is free to slide along the sides 234 and 236 of the upper support portion 214 such that a bottom surface 208 moves toward the top surface 204. In one arrangement, the v-shaped notches 226 and 244 align. When the second plate 206 is slid upwardly, the space between the first plate 202 and the second plate 206 increases allowing the universal needle guide 210 to support a larger needle gauge. When the second plate 206 is slid downwardly, the universal needle guide 210 is capable of supporting a smaller needle gauge. Although FIG. 4 illustrates a first plate 202 and a second plate 206 both slidably engaged with the upper support portion 214, first plate 202 could be stationary. In one embodiment, the second plate 206 may include at least one locking tab 216 on at least one side 218 of second plate 206 to lock second plate 206 in a specific position. The locking tab 216 is positioned complementary to a plurality of notches 220 on at least one of side surfaces 234 and 236; however, other types of locking mechanisms could be used.

Other embodiments of the universal needle guide 210 may include various shaped openings or an opening in only one of the first plate 202 or second plate 206 so long as the biopsy needle is properly aligned between and supported by the plates to prevent movement during deployment.

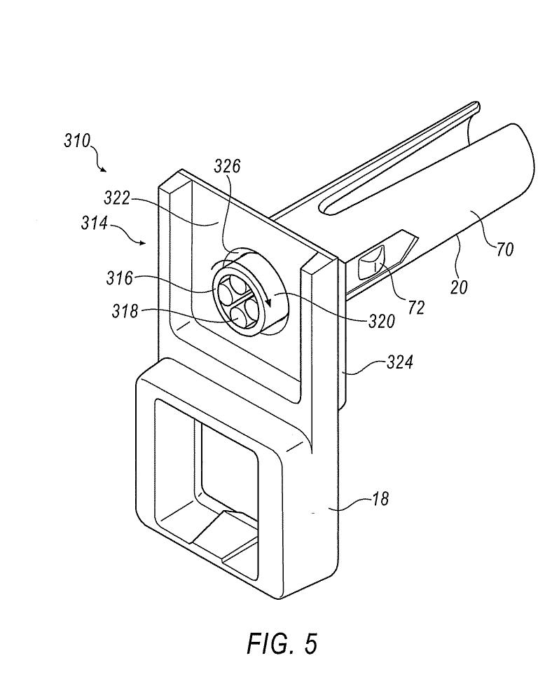

FIG. 5 illustrates a universal needle guide 310 rotatably compatible with multiple needle gauges. The upper support portion 314 has a front surface 322, a back surface 324, and a needle aperture 326 extending therethrough. The upper support portion 314 also includes a chuck unit comprising a barrel 316 disposed within the needle aperture 326, at least one clamping rods 318 positioned within the barrel 316, and a rotary collar 320 raised around the periphery of the barrel 316. A biopsy needle can be inserted into the axial center of the barrel 316 and the collar 320 can be turned clockwise causing the clamping rods 318 to move inwardly, thereby frictionally engaging the biopsy needle. To release the biopsy needle or increase the diameter of the needle aperture 326 to accommodate a larger needle gauge, the rotary collar 320 can be rotated counterclockwise. The relationship between the rotation of the collar 320 and the clamping rods 318 could be that of any chuck unit known to one of ordinary skill in the art. Thus, the rotary collar 320 allows the universal needle guide 310 to be compatible with various needle gauges by allowing the user to selectively narrow or increase needle aperture 326.

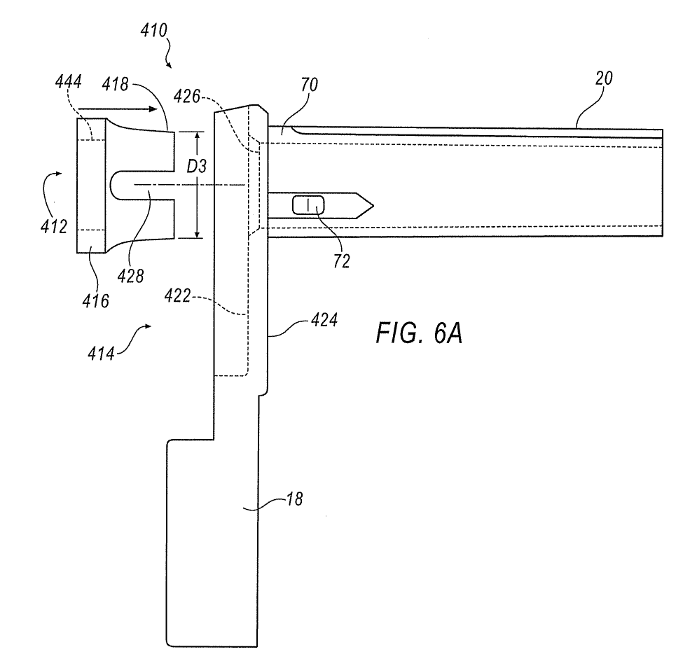

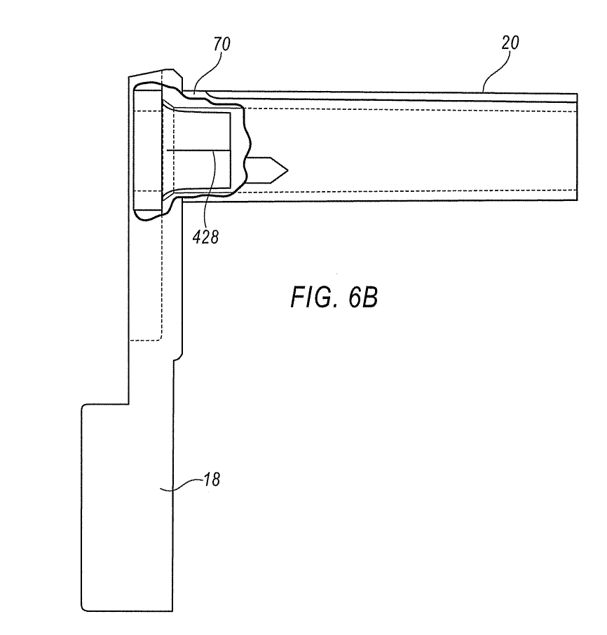

FIGS. 6A and 6B illustrate a universal needle guide 410 having a plug 412 that is slidably engaged with a first needle aperture 426. Like previous embodiments, the upper support portion 414 has a front surface 422, a back surface 424, and the first needle aperture 426 extends therethrough. The plug 412 has a head 416 containing a second needle aperture 444 and pair of flexible legs 418 configured to facilitate alignment of the biopsy needle. FIG. 6A shows an exploded view of the plug 412 and the universal needle guide 410.

In this embodiment of the universal needle guide 410, the plug 412 has a non-actuated configuration and an actuated configuration. In the non-actuated configuration, the legs 418 extend inwardly from an outer surface of head 416 a predetermined length. The legs 418 define a diameter D3 that is at least slightly larger than the first needle aperture 426. Because the plug 412 is not actuated, a space 428 remains between the legs 418 facilitating alignment of a larger gauge biopsy needle. A biopsy needle (not shown) is inserted into the second needle aperture 444 while the plug 412 is in a non-actuated configuration.

To move the plug 412 into the actuated configuration shown in FIG. 6B, the legs 418 of the plug 412 are slidably engaged with the first needle aperture 426 such that the sidewalls defining the first needle aperture 426 force the flexible legs 418 toward on another, thereby compressing the space 428. By compressing the space 428, the flexible legs 418 are forced to grip around the biopsy needle when the plug 412 has been pushed inwardly and is fully seated within the first needle aperture 426. As shown in FIG. 6B, the head 416 of the plug 412 is larger than the inner diameter of the first needle aperture 426 such that when the head 416 is pushed inwardly, it may abut the front surface 422, but it will never be disposed within the first needle aperture 426. The forward movement of the plug 412 forces the legs 418 further into the first needle aperture 426 causing a reduction in the space 428 between the legs 418 allowing the universal needle guide 410 to accommodate a smaller gauge biopsy needle.

Although certain preferred embodiments of the present disclosure have been described, the disclosure is not limited to the illustrations described and shown herein, which are deemed to be merely illustrative of the best modes of carrying out the disclosure. A person of ordinary skill in the art will realize that certain modifications and variations will come within the teachings of this disclosure and that such variations and modifications are within its spirit and the scope as defined by the claims.

What is claimed is:

1. A universal needle guide comprising:
a body having an upper support portion and at least one attachment portion;
a first needle aperture defining a first diameter disposed in the upper support portion; and
a second needle aperture defining a second diameter that is selectively alignable with the first needle aperture;
wherein the second diameter is less than the first diameter.

2. The universal needle guide of claim 1, wherein the attachment portion is a lower attachment portion extending downwardly from the upper support portion and having a peripheral edge defining an opening.

3. The universal needle guide of claim 1, wherein the attachment portion is a lower attachment portion extending downwardly from the upper support portion and having a cradling aperture.

4. The universal needle guide of claim 1, wherein the attachment portion is a slitted cannula aligned with the first needle aperture and extending outwardly from a back surface of the upper support portion.

5. The universal needle guide of claim 4, wherein the slitted cannula is spring loaded and has an outer surface with a release mechanism disposed thereon.

6. The universal needle guide of claim 1, wherein the first needle aperture extends through the upper support portion.

7. The universal needle guide of claim 1, wherein the second needle aperture extends through a faceplate.

8. The universal needle guide of claim 7, wherein a sleeve is disposed around the periphery of the second needle aperture.

9. The universal needle guide of claim 7, wherein the faceplate is pivotally attached to the upper support portion such that the first needle aperture and the second needle aperture align when the upper support portion and the faceplate are mated.

10. The universal needle guide of claim 7, wherein the faceplate is slidably attached to the upper support portion such that the first needle aperture and the second needle aperture align when the upper support portion and the faceplate are mated.

11. The universal needle guide of claim 1, wherein the at least one attachment portion is configured to selectively secure the needle guide in at least two directions.

12. The universal needle guide of claim 11, wherein the body includes at least two attachment portions, wherein a first attachment portion is configured to selectively secure the needle guide in at least a first direction and a second attachment portion is configured to selectively secure the needle guide in at least a second direction that is different than the first direction.

13. A universal needle guide comprising:
a body having an upper support portion and at least one attachment portion;
a first plate configured within the upper support portion and having a first notch extending downwardly from a top surface of the first plate; and
a second plate slidably engaged with the upper support portion;
wherein the second plate may be selectively slid toward the first plate to adjust the size of a needle that may be accommodated by the notch.

14. The universal needle guide of claim 13, wherein the attachment portion is a lower attachment portion extending downwardly from the upper support portion and having a peripheral edge defining an opening.

15. The universal needle guide of claim 13, wherein the attachment portion is a lower attachment portion extending downwardly from the upper support portion and having a cradling aperture.

16. The universal needle guide of claim 13, wherein the attachment portion is a slitted cannula aligned with the first needle aperture and extending outwardly from a back surface of the upper support portion.

17. The universal needle guide of claim 16, wherein the slitted cannula is spring loaded and has an outer surface with a release mechanism disposed thereon.

18. The universal needle guide of claim 13, wherein the first notch is substantially v-shaped.

19. The universal needle guide of claim 13, wherein the second plate has a second notch extending upwardly from a bottom surface of the second plate such that the first notch and second notch are alignable.

20. The universal needle guide of claim 19, wherein the second notch is substantially v-shaped.

21. The universal needle guide of claim 13, wherein the second plate includes at least one locking tab on at least one side surface of the second plate, the locking tab being positioned complementary to a plurality of notches on at least one side surface of the upper support portion.

22. A universal needle guide comprising:
a body having an upper support portion and at least one attachment portion;
a needle aperture disposed in the upper support portion; and
a needle retaining mechanism selectively configurable to secure multiple needle sizes.

23. The universal needle guide of claim 22, wherein the needle retaining mechanism is a rotary collar.

24. The universal needle guide of claim 23, wherein the rotary collar further includes at least one clamping rod configured to move inwardly when the rotary collar is rotated.

25. The universal needle guide of claim 24, wherein the at least one clamping rod frictionally engages a biopsy needle.

26. The universal needle guide of claim 22, wherein the needle retaining mechanism is a push actuator having a head and a pair of flexible legs extending inwardly from the head.

27. The universal needle guide of claim 26, wherein the push actuator is slidably engaged with the first needle aperture such that the flexible legs move inwardly when the push actuator is pushed towards the first needle aperture.
